(19) United States
(12) Patent Application Publication
(10) Pub. No.: US 2012/0184941 A1
(43) Pub. Date: Jul. 19, 2012

(54) MULTICOMPONENT MAGNETIC NANOPARTICLE DELIVERY SYSTEM FOR LOCAL DELIVERY TO HEART VALVE LEAFLETS AND OTHER ANIMAL TISSUES

(75) Inventors: Robert J. Levy, Merion Station, PA (US); Michael Chorny, Philadelphia, PA (US); Jeanne M. Connolly, Philadelphia, PA (US)

(73) Assignee: The Children's Hosital of Philadelphia, Philadelphia, PA (US)

(21) Appl. No.: 13/384,509

(22) PCT Filed: Jul. 21, 2010
(86) PCT No.: PCT/US10/42708

§ 371 Date: Apr. 4, 2012

Related U.S. Application Data
(60) Provisional application No. 61/227,135, filed on Jul. 21, 2009.

(51) Int. Cl. A61M 25/00; A61M 37/00

(52) U.S. Cl. 604/522; 604/264; 604/500

(57) ABSTRACT

The invention features devices, systems, and methods for targeted delivery of therapeutic agents in magnetic particle carriers to desired locations on tissue in the body. The systems and methods utilize at least one device comprising a source of magnetization, and at least one device comprising a magnetic or magnetizable material, to facilitate close tissue apposition and sealing, and to facilitate site-specific delivery of magnetic particles comprising therapeutic agents.

### RELATED APPLICATIONS

This application claims priority of U.S. provisional application 61/227,135, filed Jul. 21, 2009, the entirety of which is incorporated herein by reference.

### BACKGROUND OF THE INVENTION

Heart valve disease affects millions of individuals. Current treatments for diseased heart valves are limited to cardiac surgery to repair the valve or to replace the valve with a prosthetic. Indeed, the outcomes of both approaches are suboptimal. A need exists for less invasive treatments and therapies for heart valve disease.

### FIELD OF THE INVENTION

This invention relates generally to the field of targeted therapeutics. More specifically, the invention relates to the use of devices, including but not limited to catheters, to deliver therapeutic agent-containing magnetic nanoparticles locally to specific locations in the body, including soft tissue and heart valves.

### SUMMARY OF THE INVENTION

In one aspect, the invention provides a system for targeted delivery of a therapeutic agent to an animal tissue, including a particle including at least one therapeutic agent and a magnetic or magnetizable material, a first device including a source of magnetization, and a second device configured to release the particle.

In a further aspect, the invention provides a catheter including a proximal end, a distal end, and a shaft extending from the proximal end to the distal end, the shaft including at least one lumen extending from the proximal end to the distal end, wherein the distal end includes a magnetic or magnetizable material.

In yet another aspect, the invention provides a catheter including a proximal end, a distal end, and a shaft extending from the proximal end to the distal end, wherein the distal end includes a source of magnetization.

In still another aspect, the invention provides a method for targeted delivery of a therapeutic agent to an animal tissue, including the steps of:

(a) positioning a first device including a source of magnetization at a desired location in the tissue;

(b) positioning a second device adjacent to the source of magnetization; and,

(c) releasing a particle including the therapeutic agent and a magnetic or magnetizable material from the second device, wherein the source of magnetization attracts the particle to the desired location in the tissue.

In another aspect, the invention provides a method for targeted delivery of a therapeutic agent to a heart valve leaflet in vivo, including the steps of:

(a) contacting a desired location on the heart valve leaflet with a first device including a source of magnetization;

(b) contacting an adjacent location on the heart valve leaflet with a second device including a distal end including a magnetic or magnetizable material; and,

(c) releasing a particle including the therapeutic agent and a magnetic or magnetizable material from the second device, wherein the source of magnetization attracts the particle to the desired location in the heart valve leaflet.

### BRIEF DESCRIPTION OF THE DRAWINGS

FIG. 1 is a schematic representation of local delivery of magnetic nanoparticles to an aortic valve leaflet, using a two-catheter MNP-based delivery system according to one aspect of the invention.

FIGS. 2A-2C illustrate three exemplary embodiments of double-lumen catheters according to some aspects of the invention.

FIG. 3 shows data from treatment of an ovine aortic valve leaflet with magnetic nanoparticles containing Ad-luciferase directed to the leaflet with a magnet according to the invention, compared with a background (negative GFP control) and treatment with the nanoparticles in the absence of a magnet.

FIG. 4A shows data resulting from magnetically guided delivery of MNP-loaded BAEC to bioprosthetic heart valve leaflets, one day after delivery.

FIG. 4B shows data resulting from magnetically guided delivery of MNP-loaded BAEC to bioprosthetic heart valve leaflets, one and two days after delivery.

### DETAILED DESCRIPTION OF THE INVENTION

Various terms relating to the systems, methods, and other aspects of the present invention are used throughout the specification and claims. Such terms are to be given their ordinary meaning in the art unless otherwise indicated.

As used in this specification and the appended claims, the singular forms “a,” “an,” and “the” include plural referents unless the content clearly dictates otherwise. Thus, for example, reference to “a particle” includes a combination of two or more particles, and the like.

The term “therapeutic agent” as used herein is intended to refer to any substance or material that provides any type of benefit to the animal to which it is administered. For example, the therapeutic agent can be a pharmaceutical, biomolecule, or cell such as an endothelial cell. Tissue to be treated can be a soft tissue, and preferably can be a heart valve leaflet. The methods can be carried out on any animal, preferably a mammal, and more preferably a human being.

Except when noted, “subject” or “patient” are used interchangeably and refer to any animal, but preferably refer to mammals such as humans and non-human primates, as well as companion, farm, or experimental animals such as rabbits, dogs, cats, rats, mice, horses, cows, pigs, and the like. Humans are most preferred.

It has been observed in accordance with the present invention that magnetic nanoparticles and regionally applied magnetic gradients can be used to facilitate local delivery of therapeutic agents and site-specific treatment of diseased heart valve leaflets. Accordingly, aspects of the invention feature systems, devices, and methods for targeted delivery of therapeutic agents.

The invention provides methods for targeted delivery of a therapeutic agent to an animal tissue. Generally, the methods comprise positioning a first device comprising a source of magnetization at a desired location on the tissue, positioning a second device adjacent to the source of magnetization, and releasing a particle comprising the therapeutic agent and a magnetic or magnetizable material from the second device. Upon release of the particle, the source of magnetization attracts the particle to the desired location in the tissue. Optionally, the method steps can be repeated at the same or multiple locations on the tissue.

Either the first device, the second device, or both can directly contact the tissue, although in some aspects, neither device contacts the tissue. In preferred aspects, both devices contact the tissue, and the tissue is sandwiched between the first and second devices. The distal end of the second device can comprise a magnetic or magnetizable material. The first and/or second devices may be catheters.

In some detailed aspects, the methods can comprise contacting a desired location on a heart valve leaflet with a first device comprising a source of magnetization, contacting an adjacent location on the heart valve leaflet with a second device comprising a distal end comprising a magnetic or magnetizable material, and releasing a particle comprising the therapeutic agent and a magnetic or magnetizable material from the second device. The heart valve leaflet may be sandwiched between the first and second devices, and the first and/or second devices may be catheters. Upon release of the particle, the source of magnetization attracts the particle to the desired location in the tissue. Optionally, the method steps can be repeated at the same or multiple locations on the tissue. The heart valve leaflet can be in any animal, preferably a mammal, and more preferably a human being.

The invention also features systems for targeted delivery of a therapeutic agent to an animal tissue. The systems can comprise a particle comprising at least one therapeutic agent such as a pharmaceutical, biomolecule, or cell, and a magnetic or magnetizable material, a first device comprising a source of magnetization, and a second device configured to release the particle. Preferably, the first and second devices comprise catheters. The systems can further comprise at least one animal tissue such as a soft tissue, or a heart valve leaflet.

The distal end of the second device can comprise a magnetic or magnetizable material such as steel. The second device can also comprise at least one compartment configured to contain the particle until the particle is released.

The invention also features devices, including catheters. The catheters can comprise a proximal end, a distal end, and a shaft extending from the proximal end to the distal end. The shaft can comprise at least one lumen extending from the proximal end to the distal end of the catheter, and the distal end of the catheter can comprise a magnetic or magnetizable material such as steel. The catheter can also comprise a guidewire lumen extending from the proximal end to the distal end of the catheter. The catheter can also comprise at least one compartment configured to contain and release a therapeutic agent.

The catheters can comprise a proximal end, a distal end, and a shaft extending from the proximal end to the distal end. The distal end of the catheter can comprise a source of magnetization. The catheters can optionally further comprise at least one lumen extending from the proximal end to the distal end of the catheter. The lumen may be a guidewire lumen.

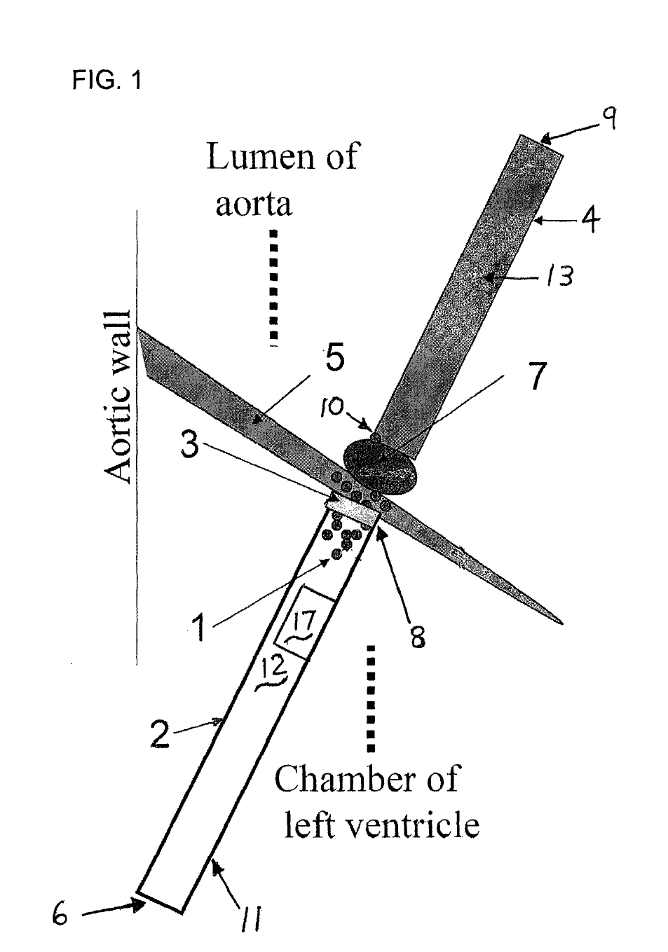

One embodiment that can be used in treating a diseased heart valve leaflet is illustrated in FIG. 1. FIG. 1 shows local delivery of magnetic nanoparticles 1 to the aortic valve leaflet 5 with a ventricular catheter 2 positioned under the ventricular surface of the aortic valve leaflet 5 in close proximity to the surface. A magnetic catheter 4 comprising a shaft 13 having a proximal end 9 and a distal end 10 has a magnetic tip 7 positioned on, or integral with, the distal end 10. Magnetic tip 7 is positioned at the outflow/aortic surface of aortic valve leaflet 5, also in close proximity to the ventricular surface. Ventricular catheter 2 has a hollow tubular shaft 11 defining a lumen 12 extending from distal end 8 to proximal end 6. Ventricular catheter 2 comprises a magnetic or magnetizable ring 3 (for example, steel) mounted on or integral with distal end 8, encircling the end of lumen 12. Catheter 2 may optionally include a compartment configured to contain and release a therapeutic agent. Such a compartment, shown schematically at 17 in FIG. 1, may internal or external to shaft 11. The positioning of the catheters 2, 4 in this delivery system can be guided either by three dimensional echo-cardiography or other imaging techniques such as fluoroscopy or computerized tomography.

Shaft 13 of magnetic catheter 4 may be solid, or it may enclose a lumen as described above for catheter 2. Shaft 13 may for example comprise a guidewire lumen though which a guidewire may be threaded when used in vivo, and the magnetic tip 7 may be in the form of a ring enclosing the distal end of the lumen so that a guidewire may pass through.

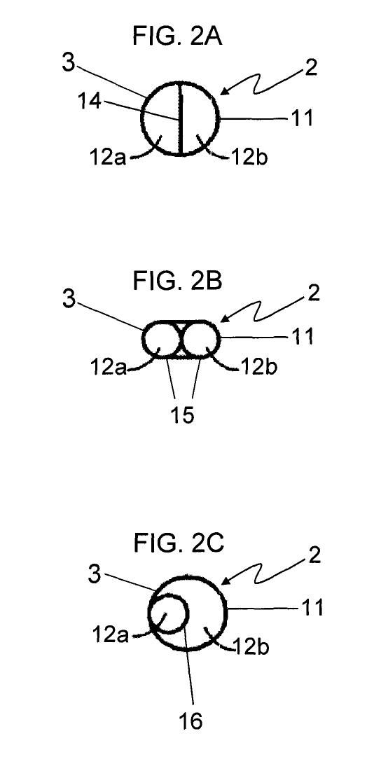

Catheter 2 may be a double-lumen catheter, examples of which are shown in FIGS. 2A-2C. FIG. 2A is an end view of an exemplary catheter 2 viewed from the proximal end. Shaft 11 is divided into two lumens 12a and 12b, separated by a longitudinal internal wall 14. A magnetic or magnetizable ring 3 as illustrated in FIG. 1 is affixed to or integral with the distal end of shaft 11, encircling the end of lumens 12a and 12b.

FIGS. 2B and 2C are end views of two other exemplary embodiments of double-lumen catheters according to the invention, viewed from the proximal end. Each has a magnetic or magnetizable ring 3 affixed to or integral with the distal end of shaft 11, surrounding the end of lumens 12a and 12b. The embodiment of FIG. 2B comprises two side-by-side tubes 15 within shaft 11, each of the tubes defining one of lumens 12a and 12b. In FIG. 2C, a narrower tube 16 enclosing lumen 12a is attached to the inner wall of tubular shaft 11, with the region outside of tube 11 but inside of shaft 11 defining lumen 12b. In any of FIGS. 2A-2C, either lumen 12a or 12b may be a guidewire lumen into which a guidewire (not shown) may be threaded for use during in vivo treatment.

Magnetic nanoparticles can be administered during normal cardiac contraction, valvular function, and circulation, and can be administered to one leaflet at a time. The catheters are designed with sufficient elasticity and other mechanical enhancements to maintain contact with the leaflets during the cardiac cycle with active contraction of the ventricular musculature. Mechanical enhancements include design features of the catheters that permit them to maintain stable contact with the surface of a heart valve leaflet even though the leaflet is moving and undergoing changes in shape during the cardiac cycle. Enhancements can include shock absorbing catheter modifications, such as bellows as shown in U.S. Pat. No. 4,886,502, or a torquable helical coil as shown in U.S. Pat. No. 6,290,656, the contents of both patents incorporated by reference herein in their entireties. The enhancement may also include a hydraulic shock-absorbing chamber. Such enhancements can allow a range of motion of the magnetically positioned catheters while the tissue between the catheters, such as a heart valve, continues its functional motion.

Ventricular catheter 2 may be tubular, with the walls of the tube defining a compartment that can be loaded with a suspension of magnetic particles. Delivery of the particles can be magnetically driven from ventricular catheter 2 onto the ventricular surface of aortic valve leaflet 5, guided by a magnet 7 juxtaposed on the other side of the leaflet.

Magnetic catheter 4 in contact with leaflet 5 on the aortic side attracts the magnetic nanoparticles into the interstices of the leaflet. In addition, magnetic catheter 4 can serve as a magnetic trap for particles not retained by the leaflet that could otherwise travel to non-targeted tissue. A steel ring 3 in the tip of delivery catheter 2 creates a tight tissue seal on both sides of the heart valve leaflet, thereby optimizing local delivery and minimizing non-targeted particle release.

Multiple magnetic nanoparticle administrations to the leaflet may be required. Nanoparticles may be delivered to the same location multiple times, and/or delivered to different locations multiple times, over a period of time. Systems, devices and methods in accordance with the invention can be used for localized delivery of therapeutic agents to any tissue. The delivery systems are preferably applied to a diseased area of an organ with a cavity. The tissue of the diseased organ can thereby function as a limiting membrane for the nanoparticles, providing a targeting site with retention properties that can either be inherent or specifically designed. For example, in the heart valve example described above, the challenges of local delivery to a dynamic heart valve leaflet in the presence of high shear blood flow and associated cardiac contractile activity must be considered.

Other soft tissue-organ cavity environments, such as retinal, joint, tendon sheath, central nervous system, gastro-intestinal tract, genito-urinary system and the cardiac chambers, can benefit from the inventive systems and methods. Access for the delivery via this approach can occur, for example, through a number of routes involving catheters, fiber-optic endoscopes (for intestine, bronchi, gall-bladder, joints, and the like), trans-ocular delivery systems, syringes, and various types of probes.

Thus, aspects of the invention feature systems for targeted delivery of a therapeutic agent to an animal tissue. In general, the systems comprise a particle comprising at least one therapeutic agent and a magnetic or magnetizable material, a first device comprising a source of magnetization, and a second device configured to release the particle.

In some aspects, the particles comprise at least one therapeutic agent and a magnetic or magnetizable material. Preferably, the particle is a nanoparticle. Magnetic nanoparticles (MNP) include particles that are permanently magnetic and those that are magnetizable upon exposure to an external magnetic field, but are no longer magnetic when the field is removed. Materials that are magnetic or magnetizable upon exposure to a magnetic field that lose their magnetic properties when the field is removed are referred to herein as superparamagnetic material. Superparamagnetic particles can be used to prevent irreversible aggregation of the particles. Examples of suitable superparamagnetic materials include, but are not limited to, iron, mixed iron oxide (magnetite), or gamma ferric oxide (maghemite) as well as substituted magnetites that include additional elements such as zinc.

Superparamagnetic material can be in the form of one or more nanocrystals, for example, single-domain crystalline systems with at least one dimension ≦100 nm. A nanocrystal is any nanomaterial with at least one dimension ≦100 nm and that is singlecrystalline or monocrystalline, or formed of a single crystal-unit such that all elements have identical crystallographic orientation of c- and a-axes and overgrow as one unit. Any particle that exhibits crystalline structure can be termed nanoparticle or nanocluster based on the dimensions of the particle.

In some aspects, the particle is a composite nanocrystal. The composite nanocrystal can comprise more than one individual magnetic or magnetizable nanocrystal and one or more water-insoluble biocompatible materials to hold the crystals together. The biocompatible material can be one or more polymers, including those described or exemplified herein.

The particle can comprise a polymer, which can be biodegradable or non-biodegradable. Non-limiting examples of such polymers include poly(urethane), poly(ester), poly(lactic acid), poly(glycolic acid), poly(lactide-co-glycolide), poly(E-caprolactone), poly(ethyleneimine), poly(styrene), poly(amide), rubber, silicone rubber, poly(acrylonitrile), poly(acrylate), poly(methacrylate), poly(a-hydroxy acid), poly(dioxanone), poly(orthoester), poly(ether-ester), poly(lactone), poly(alkylcyanoacrylate), poly(anhydride), poly(ethylenvinyl acetate), poly(hydroxybutyrate), poly(tetrafluoroethylene), poly(ethylene terephthalate, polyoxyethylene, polyoxyethlkyene-polyoxypropylene block copolymers, mixtures thereof and copolymers of corresponding monomers.

Polymeric nanoparticles, including those having incorporated superparamagnetic nanocrystals, can be prepared according to any means suitable in the art.

In some preferred aspects, the particles are bioresorbable nanoparticles, including those prepared without the use of high energy dispersion or organic solvents. Bioresorbable nanoparticles can be comprised of at least one anionic lipid salt, at least one therapeutic agent, and at least one magnetic or magnetizable material.

Bioresorbable nanoparticles can be rendered magnetic through inclusion of magnetically responsive nanocrystals in their structure, for example, by combining a fine suspension of such crystals (a ferrofluid) with the anionic lipid solution prior to the particle formation. Ferrofluids are composed of nanosacle ferromagnetic particles suspended in a carrier fluid, such as water. Preparation of such nanoparticles is a two-step process consisting of 1) making the fine suspension of magnetic nanocrystals (ferrofluid) in the presence of an anionic lipid, and 2) forming nanoparticles by controlled precipitation of the anionic lipid with a polyvalent cation in the presence of a stabilizer and a therapeutic agent. In one aspect, the magnetic nanoparticles are prepared by controlled aggregation of an oleate-stabilized ferrofluid with Ca+2.

To prepare a ferrofluid, an aqueous solution containing a water soluble ferric (Fe+3) salt, such as ferric chloride hexahydrate, and a water soluble ferrous salt (Fe+2), such as ferrous chloride tetrahydrate, is precipitated with base, such as an aqueous sodium hydroxide solution to form a magnetite precipitate containing magnetic nanocrystals. A water soluble salt of a fatty acid, such as an aqueous solution of sodium oleate, is added, and the magnetic nanocrystals are resuspended by heating, for example, in an inert atmosphere, such as under argon. A stabilizer such as albumin can be added, along with the therapeutic agent, either to the first aqueous solution, which comprises the magnetic nanocrystals, stabilizer, water soluble salt of a mono-carboxylic fatty acid, and therapeutic agent, or to the second aqueous solution, which comprises the polyvalent biocompatible cation. The second solution is then added to form the magnetic nanoparticles.

In some aspects, the therapeutic agent can be attached or tethered to the surface of a pre-formed particle or nanoparticle. The attachment can be according to any means suitable for the therapeutic application to which the agent will be used, or according to the chemical properties of the agent or the nanoparticle. For example, attachment can be by adsorption, electrostatic interactions, charge complexation, ionic bonding, or covalent bonding, and can include the use of biomolecule tethers.

The magnetic nanoparticles associated with the therapeutic agent can range in size from about 50 to about 500 nm. The size can vary according to the needs of the investigator or medical practitioner. Preferably, the nanoparticles range in size from about 50 nm to about 300 nm, and more preferably from about 100 nm to about 300 nm.

Therapeutic agents include any molecule that can be associated with a particle and used in the systems and methods of the present invention. They can be purified molecules, substantially purified molecules, molecules that are one or more components of a mixture of compounds, or a mixture of a compound with any other material. The molecules can be organic or inorganic chemicals, radioisotopes, pharmaceutical compounds, pharmaceutical salts, pro-drugs, or biomolecules, and all fragments, analogs, homologs, conjugates, and derivatives thereof. Biomolecules include, without limitation, proteins, polypeptides, nucleic acids, lipids, polysaccharides, monosaccharides, and all fragments, analogs, homologs, conjugates, and derivatives thereof. Agents can also be an isolated product of unknown structure, a mixture of several known products, or an undefined composition comprising one or more compounds. Examples of undefined compositions include cell and tissue extracts, growth medium in which prokaryotic, eukaryotic, and archaebacterial cells have been cultured, fermentation broths, protein expression libraries, and the like. Therapeutic agents can be provided in or otherwise associated with a carrier such as a pharmaceutically acceptable carrier.

For the examples described below, viral gene vectors encoding reporter proteins were delivered via catheter. Transgene expression in these studies indicates not only local delivery of magnetic nanoparticles, but tissue entry, cellular processing, nuclear pore transit and expression of transgene within the nucleus. Translational activity is evidenced by production of protein encoded by the transgene.

Thus, the therapeutic agent also can be one or more viral vector systems, which are used in gene therapy. Viral vector systems include but are not limited to adenovirus, adeno-associated virus, retrovirus and Herpes simplex virus. One of the most successful ways of introducing the gene of interest into the appropriate cell line is via recombinant adenovirus. Adenoviruses are non-enveloped particles having a diameter of about 70 nm that contain a linear double stranded DNA of approximately 36,000 base pairs. They are easily prepared with high titers and can infect a wide range of cells, including non-dividing cells. Recombinant adenovirus can also be used s in vaccination by expressing a gene product that triggers an immune response.

Adeno-associated viruses have a particle diameter of 20 nm. Retroviruses are spherical, enveloped particles having a particle diameter of between about 80 nm to about 100 nm in diameter. Retroviruses have been widely used as vectors for DNA delivery. Herpes simplex viruses have a particle diameter of about 100 nm, and contain enveloped, double-stranded DNA virus of approximately 150,000 base pairs. These viruses have a large loading capacity for foreign genes and are able to infect a wide range of cells. In addition, the virus genome remains episomal after infection, thus eliminating the possibility of opportunistic malignant insertional mutagenesis of the host genome.

Multiple agents can be included in a particle. Multiple particles comprising different therapeutic agents can also be used. Those of skill in the art can determine the particular combination of agents, based, for example, on the condition being treated, or on the needs of the particular subject. For example, additional agents that modulate the activity of a primary agent, reduce pain, support growth of therapeutic cells, are antithrombogenic, anti-apoptotic, anti-inflammatory, immunosuppressant, or antioxidant, or other agents ordinarily used in the art to treat the disease of interest can be used.

The therapeutic agents can also be formulated in sustained-release vehicles or depot preparations. For example, the agents can be formulated with suitable polymeric or hydrophobic materials (for example, as an emulsion in an acceptable oil) or ion exchange resins, or as sparingly soluble derivatives, for example, as a sparingly soluble salt. Liposomes and emulsions are well-known examples suitable for use as carriers for hydrophobic drugs.

Agents can also be one or more cells, including eukaryotic or prokaryotic cells, including stem cells such as postpartum derived cells or bone marrow derived cells, and progenitor cells. For example, the cell can be a Blood Outgrowth Endothelial Cell (BOEC), adult and cord blood stem cells (CBSC), or Induced Pluripotent Stem Cells, e.g., skin cells that are programmed to transform into pluripotent stem cells with further potential to differentiate into cells with at least one endothelial phenotype.

Non-limiting examples of agents that can be used include Nitric Oxide (NO) donors, antimicrobial agents, 3-hydroxy-3-methylglutaryl-coenzyme A reductase inhibitors (statins), antiarrhythmic agents, anticoagulants, platelet inhibiting agents and thrombolytic agents, anticalcification agents, and the like.

Non-limiting examples of suitable NO donors include B-NOD, diazeniumdiolates, molsidomine, linsidomine, S-nitrosothiols, and NO releasing non-steroidal anti-inflammatory drugs, as well as plasmid DNA or viral vectors encoding endothelial or inducible nitric oxide synthases. Non-limiting examples of antimicrobial agents include streptomycin, gentamicin, netilmicin, kanamycin, tobramycin, amikacin, rifampin, penicillin G, ceftriaxone, vancomycin, and amphotericin B. Non-limiting examples of statins include atorvastatin, rosuvastatin, simvastatin, lovastatin, and pravastatin. Non-limiting examples of antiarrhythmic agents include propafenone, flecainide, sotalol, dofetilide, amiodarone, and metoprolol. Non-limiting examples of anticoagulants, platelet inhibiting agents and thrombolytic agents include acenocoumarol, dipyridamole, clopidogrel, urokinase, and NO-aspirin. Non-limiting examples of anticalcification agents include alendronate, clodronate, and 2-mercaptoethylidene-1,1-bisphosphonate. Other suitable agents would be expected to be known to the practitioner.

In some preferred aspects, the systems comprise at least two catheters. A first catheter comprises a source of magnetization. The source of magnetization, preferably a magnet, can be configured such that the generation of a magnetic field can be controlled. The magnet may be a permanent magnet, or it may be an electromagnet. In some embodiments of the invention, the source of magnetization can be turned on or off by the investigator or medical practitioner, or the duration of the generation of the magnetic field can be controlled or adjusted. In some embodiments, the strength of the magnetic field produced by the source of magnetization can be controlled or adjusted according to any applicable variables, including, for example, the condition of the subject, the targeted tissue, the type or amount of magnetic nanoparticle being used, and the like.

A second catheter can be used to deliver magnetic particles in proximity to the source of magnetization. In some highly preferred aspects, the second catheter comprises a magnetic or magnetizable material. The magnetic or magnetizable material, for example, a superparamagnetic material, preferably is positioned on or near the second catheter's distal end to facilitate alignment of the distal end of the second catheter with the source of magnetization. More preferably, the magnetic or magnetizable material is positioned to enable apposition of the second catheter close to the targeted tissue, and to enable a tight seal with the tissue due to the interaction with the magnetic field across the tissue (FIG. 1). The magnetic or magnetizable material can be any such material suitable in the art, and preferably is steel. In addition, the magnetic or magnetizable material may be in the form of a ring that circumscribes the distal end of the second catheter, such as ring 3 shown in FIG. 1. In some aspects, the second catheter does not comprise a magnetic or magnetizable material.

The second catheter can be pre-loaded with therapeutic particles, or can be used as a conduit through which particles are loaded and pass through after the catheter is placed at or near to the desired location in the body. For example, the second catheter can comprise at least one compartment configured to contain a therapeutic agent-containing particle such as a magnetic nanoparticle as described herein, until the particle is delivered. Such a compartment can be configured as a structural aspect of the exterior of the catheter, or can be configured as a structural aspect of the interior of the catheter, such as one or more lumens or chambers on the inside of the catheter.

Where the catheter comprises one or more lumens, the aperture on the distal end of the lumen can comprise a barrier that may include a membrane, polymer, wax, seal, and the like, to prevent particles loaded into the lumen from being released through the aperture. The magnetic field can then be used to pull the magnetic particles through the barrier, or the barrier could otherwise dissolve or melt upon exposure to the body or be mechanically dislodged to release the particles at the desired location on the tissue.

In some aspects, the systems further comprise at least one tissue of an animal. The tissue can be considered a component of certain embodiments of a system because the interposition of the tissue can govern the conditions surrounding the localization of the catheters. The tissue can be any tissue in the body. In some preferred aspects, the tissue is soft tissue. In some preferred aspects, the tissue is a heart valve leaflet.

In some aspects, the systems can further comprise a retrieval system to capture and contain therapeutic particles that do not embed in or adhere to the target site, or to capture and contain particles after the therapeutic agent has been delivered to the target site, i.e., spent particles. To minimize risks to the subject, it is preferable to remove such unused and/or spent particles. The retrieval system preferably captures and contains most, and more preferably substantially all unused and/or spent particles such that the body is substantially free of spent or unused particles. The retrieval system can be magnetic or magnetizable materials. The retrieval system can be any blood apheresis system suitable in the art. In some preferred aspects, the first catheter comprising a source of magnetization can be used as the retrieval system.

Also featured in accordance with the present invention are catheters. In some aspects, a catheter comprises a proximal end, a distal end, and a shaft extending from the proximal end to the distal end. The distal end of the catheter can comprise a magnetic or magnetizable material such as steel, or can comprise a source of magnetization. The distal end of the catheter is inserted into the body of the subject and guided to the desired location in the body according to any procedure suitable in the art.

In some preferred aspects, the catheter shaft comprises at least one lumen extending from the proximal end to the distal end of the catheter with an aperture on the proximal and distal end. The lumen can be used to load therapeutic agent-containing particles and/or deliver such particles to a desired location in the body. The lumen can also be used to house a guidewire. In some aspects, the catheter comprises a separate lumen for delivering particles, and a separate lumen for housing a guidewire. In some aspects, the catheter comprises at least one compartment configured to contain and/or release a particle comprising a therapeutic agent and a magnetic or magnetizable material. The compartment may be positioned on the exterior of the catheter and/or may be positioned on the interior of the catheter.

Catheter design possibilities at this time have great breadth and flexibility. Thus, it is possible to have multi-lumen, multi-compartment catheters that can be microprocessor-controlled and even have mechanical features for manipulating configuration and contact disposition.

Aspects of the invention also feature methods for targeted delivery of a therapeutic agent to an animal tissue. Generally, the methods comprise positioning a first device comprising a source of magnetization in proximity to a desired location in the tissue, positioning a second device adjacent to the source of magnetization, and releasing a particle comprising a therapeutic agent and a magnetic or magnetizable material from the second device. The magnetic field generated by the source of magnetization attracts the particle to the desired location in the tissue between the first device and second device.

Preferably, each device is positioned at the desired location in the tissue. Although the devices do not need to contact the target tissue, in preferred aspects, each device directly contacts the target tissue, and most preferably the contact is at the specific site on the tissue to receive the therapeutic agent in the magnetic nanoparticle carrier. In highly preferred aspects, the first and second devices contact the tissue, and the tissue is sandwiched between the distal end of each respective device.

In some aspects, the distal end of the second device comprises a magnetic or magnetizable material such as steel. This material can be in any suitable shape, including a ring or a perforated disc.

The methods can be used for targeted delivery of therapeutic agents to any cell, tissue, organ, or subpart thereof in the body. Preferably, the target tissue is a soft tissue. Most preferably, the target tissue is a heart valve leaflet. The methods can also be used for targeted delivery of therapeutic agents in vitro.

In one detailed embodiment, the methods are adapted for targeted delivery of a therapeutic agent to a heart valve leaflet in vivo, and comprise contacting a desired location on the heart valve leaflet with a first catheter comprising a source of magnetization, contacting an adjacent location on the heart valve leaflet with a second catheter comprising a distal end comprising a magnetic or magnetizable material, and releasing a particle comprising the therapeutic agent and a magnetic or magnetizable material from the second catheter. The magnetic field generated by the source of magnetization attracts the particle to the desired location in the heart valve leaflet.

The methods can optionally further comprise repeating one or more of the steps at least once. These steps can be repeated multiple times as necessary.

The following examples are provided to describe exemplary aspects of the invention in greater detail. They are intended to illustrate, not to limit, the invention.

### EXAMPLE 1

Magnetic Guided Gene Vector for Delivery to Sheep Aortic Valves in Organ Culture

In this example, Type 5 replication-defective adenoviruses, all with the human cytomegalovirus promoter, encoding green fluorescent protein (GFP), beta galactosidase or firefly luciferase were used, respectively. The obtained magnetic nanoparticles (MNP) were diluted to a final concentration of 1:1000 in cell culture medium supplemented with fetal bovine serum (10%), and added to heart valve tissue placed in a well of a 24-well cell culture plate (400 μl per well) for 30 min with/without exposure to a high gradient magnetic field generated by Nd—Fe—B magnets (1500 G magnetic flux density at the surface, 1.8 cm×1.2 cm×0.5 cm). The tissue was then washed and incubated at 37° C. in fresh serum-supplemented cell culture medium.

The initial experiments were static prototypes involving aortic valve leaflets positioned on top of a fixed magnet in cell culture media. Magnetic nanoparticles containing adenoviral vectors encoding one of three different reporter constructs were then administered to the top side of leaflets for 30 minutes, followed by exhaustive washing, followed by incubation at 37° C. in a cell culture incubator, as described below.

Magnetically responsive nanoparticles: MNPs containing adenovirus were prepared using the following two-step procedure. In the first step, nanocrystalline magnetite was obtained from ferric chloride hexahydrate and ferrous chloride tetrahydrate (170 mg and 62.5 mg, respectively) reacted with an equivalent amount of sodium hydroxide (1 M). The precipitate was magnetically separated and coated with sodium oleate (225 mg in 5 ml water) by two cycles of heating under argon to 90° C., and ultrasonication (5 min each step). In the second step, magnetic nanoparticles were formed in the presence of adenovirus (Ad, 5×1011 viral particles) and Poloxamer 407 (20 mg) by dropwise addition of an aqueous solution of zinc chloride (0.1 M, 0.75 ml) upon gentle stirring. The particles were washed twice by magnetic decantation prior to reconstitution in 5% glucose aqueous solution. The obtained MNP contained an estimated 1.4×107 pfu per μL, resulting in a typical delivery load of 1.4×107 pfu/ml in the experimental designs described herein.

The magnetic particles prepared as described above are composite particles. Nanocrystalline magnetite is formed in the first step by alkaline precipitation, and the composite particles are obtained in the second step by controlled precipitation of zinc oleate with magnetite nanocrystals and adenovirus entrapped in the particle matrix.

A. Local Delivery of MNPs Containing Adenoviral Gene Vectors Encoding Green Fluorescent Protein (GFP).

Ovine aortic valve leaflets were obtained and placed into cell culture dishes containing nutrient media. In each case a magnet (1500 Gauss) was placed underneath the culture dish, and 200 μL magnetic nanoparticles, prepared as described above, containing Ad-GFP were added to the media. After 30 minutes, the magnets were removed, and the leaflets were washed to remove exogenous particles, and placed into fresh media for continued culture under normal growth conditions. GFP expression was observed in valvular cells by fluorescent microscopy 24 hours later, demonstrating that transduction had taken place. After 7 days in culture, minimal GFP expression was observed in parallel valve leaflets which had not been exposed to a magnetic field at the time of magnetic nanoparticle exposure. However, robust GFP expression was seen to increase in valve leaflets exposed to the full multicomponent nanoparticle delivery system involving magnetic nanoparticles, containing GFP Adenoviruses with magnetic field exposure.

B. Tissue Distribution Following Magnetic Leaflet Delivery, Comparable to A (Above) with MNPs Containing Ad-Beta-Galactosidase.

Experiments duplicating the conditions described in A, above, but utilizing β-galactosidase instead of GFP as the reporter gene, were developed to display blue color upon expression of the reporter construct 24 hours after multicomponent transduction. The unmagnified gross appearance of a β-Galactosidase expressing leaflet showed blue coloration indicating that the complex tissue of the leaflet had received, retained, and biologically processed the reporter construct. Frozen sections of this tissue imaged at 100× magnification showed positive β-galactosidase-expressing cells (blue) in the central interstitial cells, indicating that the magnetically-driven nanoparticle payload is capable of being delivered into the tissue layers rather than just deposited onto the surface of the leaflet.

C. Ad-Luciferase local delivery studies as above, with magnetic nanoparticles containing Ad-luciferase, using quantitative optical imaging to demonstrate magnetically driven local delivery to heart valve leaflets.

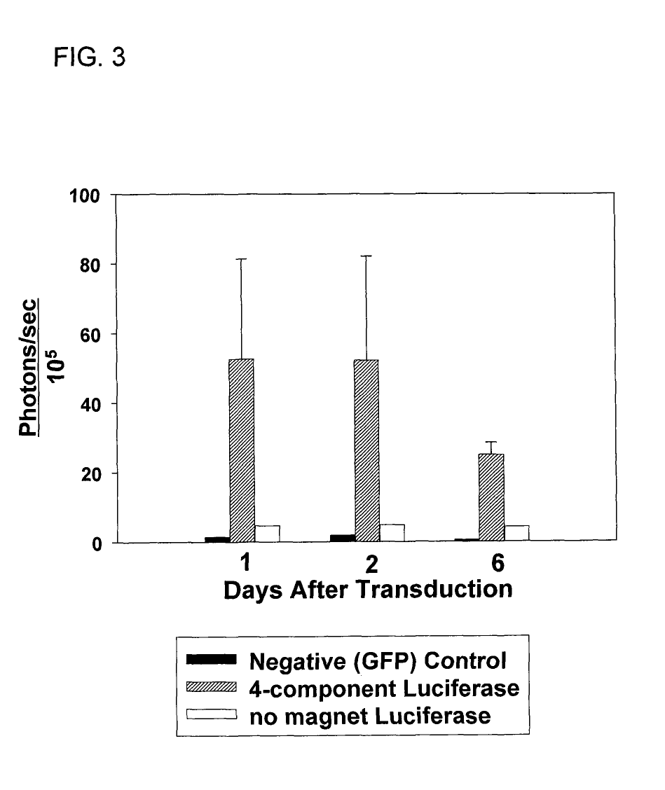

The multicomponent magnetic nanoparticle delivery system was again utilized as described in A above, but luciferase was substituted as the reporter gene. Luciferase expression was subsequently documented quantitatively at multiple timepoints, following luciferin administration, using the MS Imaging System (IVIS, Caliper Lifesciences, Hopkington, Mass.), for optical imaging and quantitative luminescence. FIG. 3 shows an example of data collected from valve leaflets sequestered in cell culture dishes using this system. Using this reporter/quantitation system, the efficacy and specificity of the magnetic component system were confirmed. As shown in FIG. 3, significantly higher expression of the adenovirus-luciferase reporter in the tissue was obtained using the complete delivery system compared to either background (negative GFP control) or delivery with no magnet. Robust cellular uptake and biological processing of the payload was demonstrated by the duration of luciferase activity.

### EXAMPLE 2

Magnetic Cell Delivery to Bioprosthetic Heart Valve Leaflets Using MNP/Magnetic Guidance with MNP Loaded Bovine Aortic Endothelial Cells (BAEC)

These experiments investigated the possibility of delivering endothelial cells to heart valve leaflets to enable regeneration of an endothelium.

MNP were loaded into BAEC that had been transduced with Ad-Luc in cell culture. Five μg of non-Ad-containing MNP were loaded per 1.5×104 BAEC cells, using BAEC transduced with 1.75×107 pfu-AdLuc/1.5×104 cells. The concentration of cells in the suspensions used for magnetic cell targeting was 105 cells/leaflet, and control Ad-luc cells were prepared in parallel without MNP loading, as previously published by Polyak et al. (2008) Proc. Natl. Acad. Sci. USA, 105:698-703.

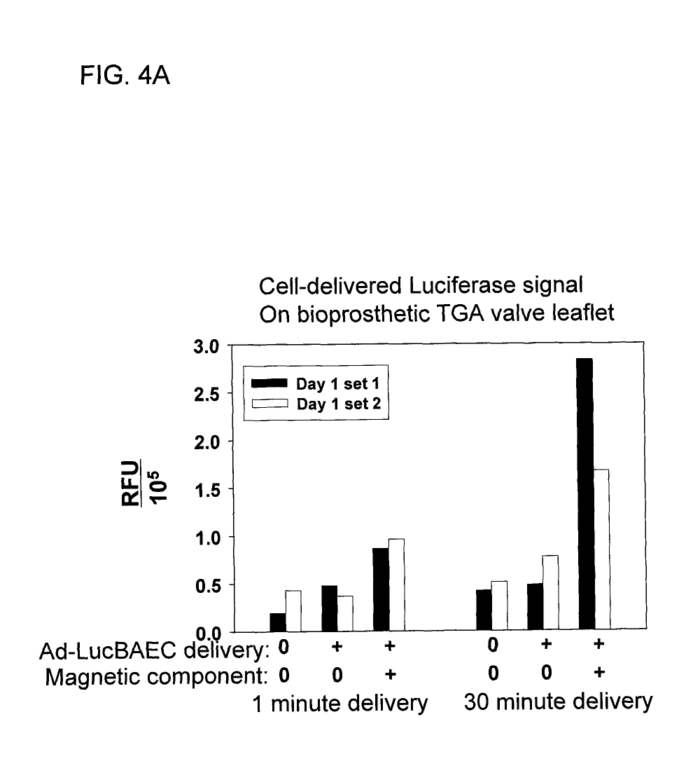

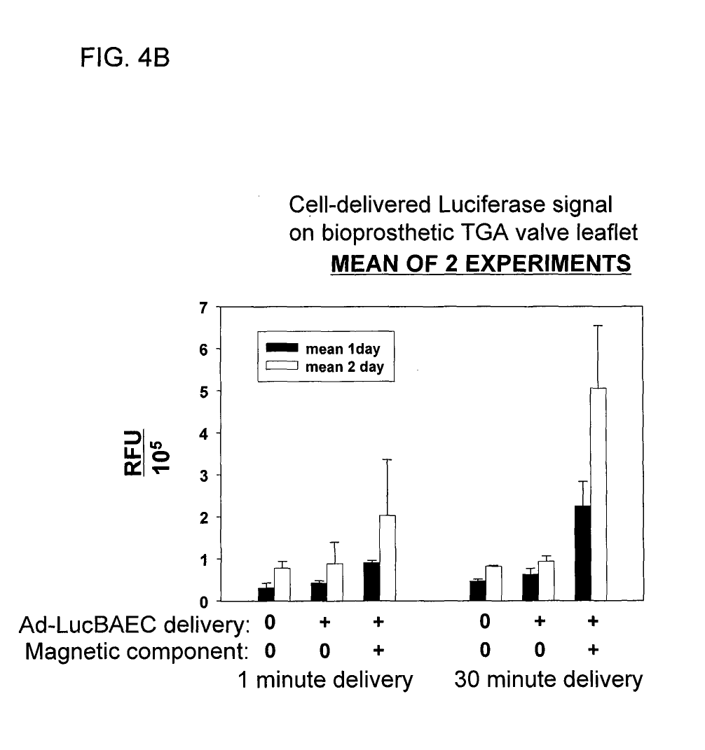

FIG. 4A shows data resulting from magnetically guided delivery of the MNP-loaded BAEC to bioprosthetic heart valve leaflets, taken one day after delivery of the particles. FIG. 4B consolidates those data with data obtained two days after delivery. In each of two duplicate runs (Set 1 and Set 2), three leaflets were located in a standard cell culture well and treated individually as follows. For the first leaflet, the MNP-loaded BAEC were locally delivered to the leaflet under the influence of a 1 cm diameter magnet (3600 Gauss) positioned underneath for either one or 30 minutes. The second leaflet was similarly treated but in the absence of a magnet, and the third leaflet was not exposed to MNP-Ad-luciferase BAEC or a magnet. The AD-Luciferase reporter was quantified using the IVIS imaging system as used in generating the data shown in FIG. 3. Significant signal was recorded from leaflets treated with the MNP-loaded cells under the influence of the magnet. The luciferase signal evident one and two days after delivery of adenoviral luciferase vector indicates that the BAEC cells remained viable and capable of processing Ad-luciferase message.

Both 1 and 30 minute magnetic exposures gave strong luciferase expression that increased in intensity from day 1 following targeting (FIGS. 4A and 4B) to day 2 (FIG. 4B). These results illustrate that even genetically engineered cells can be targeted with this tightly controlled MNP-magnetic guidance system, and indicate the possibility of significant advantage i n providing a cellular lining that would resist thrombosis and inflammatory activity.

### EXAMPLE 3

Prototype Designs of a Two-Catheter Based System for Treating Heart Valve Leaflets

A. Design Illustrations

One strategy in accordance with the invention is to provide highly localized magnetically targeted site-specific delivery to heart valve leaflets and other important therapeutic sites. This can be achieved through a catheter configuration that uses MNP with guidance based upon a delivery catheter with a steel anchoring ring around the perimeter of its tip, and a magnetic tipped catheter to position the delivery catheter in close proximity to the tissue site to be targeted. The steel ring interaction with the delivery catheter magnetic field results in a tight tissue seal to minimize downstream loss of non-targeted MNP. This system is illustrated in FIG. 1.

B. In vitro Simulations Using the Prototype Catheter-MNP Delivery System

These experiments simulate use of the two-catheter prototype system, using TYGON® tubing with a #10 lock washer positioned on the distal end of the tubing to simulate a MNP-delivery catheter. The purpose of the steel washer was to provide a magnetically attractive zone on the end of the tubing to pull the tip of the tubing into a tight sealing position with an ovine heart valve leaflet (or fresh ovine pericardial segment) positioned on top of a fixed magnet. The fixed magnet simulated a magnetic catheter tip as described above.

Experiments were carried out as follows: ovine aortic valves and pericardia were obtained fresh after euthanasia under an approved IACUC protocol. Leaflets or pericardium were dissected free of unrelated tissue and were rinsed with copious amounts of sterile saline. The prototype steel-washer tipped catheter was positioned on the upper surface of each specimen in a standard cell culture well, and a fixed cell culture magnet (1000 Gauss) was placed underneath each specimen. A suspension of nanoparticles containing Ad-Luc (estimated at 1.4×106 pfu/100 μl volume) was then added to the inner chamber of each prototype catheter and held for a predetermined length of time. A control was also performed in which the magnet was present under the specimen but no catheter was used.

Table 1 shows the results of runs under various conditions, where RFU units indicate the strength of resultant luciferase expression by the washed tissues, obtained using luciferin hydrolysis with detection using an IVIS system for quantifying optical luminescence of luciferase hydrolyzed luciferin (Luc). Each RFU figure represents the reading taken from the highest signal strength area of the treated tissue for each experimental condition. The images (not shown) from which the data were taken revealed that direct addition of MNP-AdLuc resulted in diffuse Luc expression over the treated area, while targeting MNP-AdLuc with the prototype catheter resulted in intensely focused transgene expression. Increasing exposure time to the magnet resulted in higher levels of MNP-driven transgene expression, as did delivery of increased amounts of MNP-AdLuc.

These data demonstrate proof of concept in vitro with a fully functional prototype that is comparable to the configuration that would be used in vivo for local targeting of MNP to heart valve leaflets using the complex approach described herein.

TABLE 1

| | | | | |
|---|---|---|---|---|
| | Time | μL | | |
| Catheter? | (min) | MNP | Tissue | RFU/105 |

| | | | | |
|---|---|---|---|---|
| N | 30 | 100 | leaflet | 1.80 |
| Y | 30 | 100 | leaflet | 8.15 |
| Y | 5 | 100 | pericardial | 0.23 |
| Y | 5 | 100 | pericardial | 0.08 |
| Y | 10 | 100 | pericardial | 0.07 |
| Y | 10 | 100 | pericardial | 0.90 |
| Y | 15 | 100 | pericardial | 0.89 |
| Y | 15 | 100 | pericardial | 1.13 |
| Y | 15 | 25 | pericardial | 0.15 |
| Y | 15 | 25 | pericardial | 0.18 |
| Y | 15 | 100 | pericardial | 0.47 |
| Y | 15 | 100 | pericardial | 0.71 |

The present invention is not limited to the embodiments described and exemplified above, but is capable of variation and modification within the scope and range of equivalents of the appended claims.

1. A system for targeted delivery of a therapeutic agent to an animal tissue, comprising a particle comprising at least one therapeutic agent and a magnetic or magnetizable material, a first device comprising a source of magnetization, and a second device configured to release the particle.

2. The system of claim 1, wherein the second device comprises a magnetic or magnetizable material.

3. The system of claim 2, wherein the magnetic or magnetizable material comprised by the second device is steel.

4. The system of claim 1, wherein the second device comprises at least one compartment configured to contain the particle until the particle is released.

5. The system of claim 1, wherein the at least one therapeutic agent is a pharmaceutical, biomolecule, or cell.

6. The system of claim 1, wherein the at least one therapeutic agent is an endothelial cell.

7. The system of claim 1, further comprising at least one animal tissue.

8. The system of claim 7, wherein the at least one animal tissue is a soft tissue or a heart valve leaflet.

9. (canceled)

10. The system of claim 1, wherein the first and second devices comprise catheters.

11. A catheter comprising a proximal end, a distal end, and a shaft extending from the proximal end to the distal end, the shaft comprising at least one lumen extending from the proximal end to the distal end, wherein the distal end comprises a magnetic or magnetizable material.

12. The catheter of claim 11, further comprising a guidewire lumen extending from the proximal end to the distal end.

13. The catheter of claim 11, wherein the magnetic or magnetizable material is steel.

14. The catheter of claim 11, further comprising at least one compartment configured to contain and release a therapeutic agent.

15. A catheter comprising a proximal end, a distal end, and a shaft extending from the proximal end to the distal end, wherein the distal end comprises a source of magnetization.

16. The catheter of claim 15, further comprising at least one lumen extending from the proximal end to the distal end.

17. The catheter of claim 16, wherein the at least one lumen is a guidewire lumen.

18. A method for targeted delivery of a therapeutic agent to tissue of an animal, comprising the steps of:
(a) positioning a first device comprising a source of magnetization at a desired location in the tissue;
(b) positioning a second device adjacent to the source of magnetization; and,
(c) releasing a particle comprising the therapeutic agent and a magnetic or magnetizable material from the second device, wherein the source of magnetization attracts the particle to the desired location in the tissue.

19. The method of claim 18, wherein the first device contacts the tissue.

20. The method of claim 18, wherein the second device contacts the tissue.

21. The method of claim 18, wherein the first and second devices contact the tissue, and wherein the tissue is sandwiched between the first and second devices.

22. The method of claim 18, wherein that portion of the second device nearest the source of magnetism in step (b) comprises a magnetic or magnetizable material.

23. The method of claim 18, wherein the therapeutic agent is a pharmaceutical, biomolecule, or cell.

24. The method of claim 18, wherein the therapeutic agent is an endothelial cell.

25. The method of claim 18, wherein the tissue is a soft tissue or a heart valve leaflet.

26. (canceled)

27. The method of claim 18, further comprising repeating steps (a)-(c) at least once.

28. The method of claim 18, wherein the animal is a mammal.

29. The method of claim 28, wherein the mammal is a human being.

30. The method of claim 18, wherein the first and second devices comprise catheters.

31. A method for targeted delivery of a therapeutic agent to a heart valve leaflet in vivo, comprising the steps of:
(a) contacting a desired location on the heart valve leaflet with a first device comprising a source of magnetization;
(b) contacting an adjacent location on the heart valve leaflet with a second device comprising a distal end comprising a magnetic or magnetizable material; and,
(c) releasing a particle comprising the therapeutic agent and a magnetic or magnetizable material from the second device, wherein the source of magnetization attracts the particle to the desired location in the heart valve leaflet.

32. The method of claim 31, wherein the heart valve leaflet is sandwiched between the first and second devices.

33. The method of claim 31, wherein the therapeutic agent is a pharmaceutical, biomolecule, or cell.

34. The method of claim 31, further comprising repeating steps (a)-(c) at least once.

35. The method of claim 31, wherein the first and second devices comprise catheters.
